(19) United States
(12) Patent Application Publication
(10) Pub. No.: US 2006/0095062 A1
(43) Pub. Date: May 4, 2006

(54) Universal catheter tunneler

(75) Inventor: John Stephens, Perkiomenville, PA (US)

Correspondence Address: MONTE & MCGRAW, PC, 4092 SKIPPACK PIKE, P.O. BOX 650, SKIPPACK, PA, 19474, US

(73) Assignee: Medical Components, Inc., Harleysville, PA (US)

(21) Appl. No.: 11/263,562

(22) Filed: Oct. 31, 2005

Related U.S. Application Data
(60) Provisional application No. 60/623,998, filed on Nov. 1, 2004.

(51) Int. Cl. A61M 29/00

(52) U.S. Cl. 606/191

(57) ABSTRACT

A tunneler assembly (100) for use with a catheter assembly (50), for securing the catheter assembly to a patient. The tunneler assembly includes a tunneler (102) and a connector assembly (110), the connector assembly being removably securable to the tunneler's proximal end (104) and also extending through the or one lumen (56) of the catheter to be engaged with the proximal end of the catheter assembly to push the catheter assembly forwardly through the newly created tunnel during performance of the tunneling procedure, after which the connector assembly (110) is removable to disconnect the tunneler (102) from the catheter. A connector section (114) on the proximal end (104) of the tunneler (102) enables an adapter (130) of the connector assembly to lock onto the tunneler, after which a sheath (160) is slid into position over the tunneler (102) and then over the connector assembly (110) to sheathe the connector assembly and adjacent portions of the catheter distal end (54) and the tunneler proximal end (104).

### CROSS-REFERENCE TO RELATED APPLICATIONS

This application claims priority from U.S. Provisional Application Ser. No. 60/623,998 filed Nov. 1, 2004.

### FIELD OF THE INVENTION

This invention relates to the field of medical devices and more particularly to tunnelers for use with placement of catheters.

### BACKGROUND OF THE INVENTION

Certain medical procedures involve placement of a catheter into a patient where the catheter remains for a length of time in the patient, and such catheter placement procedures are well known. For chronic use, when the catheter is intended to remain in the patient for an extended period of time, and the proximal end of the catheter remains exposed outside the patient for connection to and disconnection from other medical devices for aspiration, for infusion of fluids, or especially for hemodialysis, it is crucially important that the implanted catheter, particularly its distal end, remain stabilized precisely in position in the blood vessel against all movement resulting from stress on the proximal catheter end. Furthering this purpose, the proximal catheter end outside of the blood vessel is secured in its position to the patient by known tunneling procedures; the catheter is typically tunneled into the patient prior to inserting the catheter into the patient's blood vessel. It is known to utilize a sheath positioned over the proximal tunneler end and the distal catheter end during tunneling.

Catheters may be placed in various venous locations and cavity locations throughout the body of a patient for introduction of fluids to the body or removal of fluids from the body. Such catheterization may be performed by using a single catheter having multiple lumens, and a typical example of a multiple lumen catheter is a dual lumen catheter in which one lumen introduces fluid and the other lumen removes fluid. An example of such a multiple lumen catheter is the SPLIT STREAM™ catheter, manufactured by Medical Components, Inc. of Harleysville, Pa.

Generally, to insert any catheter into a blood vessel, the vessel is identified by aspiration with a long hollow needle in accordance with the well-known Seldinger technique. When blood enters a syringe attached to the needle, indicating that the vessel has been found, a thin guide wire is then introduced, typically through a syringe needle or other introducer device into the interior of the vessel. The introducer device is then removed, leaving the end portion of the guide wire that has been inserted into the vessel within the vessel and the opposing end of the guide wire projecting beyond the surface of the skin of the patient. At this point, several options are available to a physician for catheter placement. The simplest is to pass a catheter into the vessel directly over the guide wire until the distal tip is precisely located along the vessel utilizing imaging apparatus. The guide wire is then removed, leaving the catheter in it desired position within the vessel. However, this technique is only possible in cases where the catheter is of a relatively small diameter, made of a stiff material, and not significantly larger than the guide wire. For example, this technique may be used to insert small diameter dual lumen catheters into a patient. If the catheter to be inserted is significantly larger than the guide wire, a dilator and sheath assembly is passed over the guide wire to enlarge the hole. The guide wire and dilator are then removed, and the catheter is then inserted through the sheath and into the vessel. The sheath is then removed by peeling the sheath from around the catheter while pulling the sheath from the vessel.

### BRIEF SUMMARY OF THE INVENTION

The present invention is a tunneler assembly for use with a catheter assembly, the tunneler assembly having a tunneler and its sheath, and an adapter for removably connecting the tunneler to the catheter assembly for performing the tunneling procedure, whereafter the sheath and adapter are easily removed to disconnect the tunneler from the catheter assembly. The adapter includes an elongate portion that extends through the lumen of the catheter, or through a selected lumen of a multilumen catheter, from the proximal lumen end to and beyond the distal lumen end, where the protruding adapter distal end is coupled to the tunneler proximal end, whereafter the sheath is slid over the connection of the catheter assembly to the tunneler. The adapter proximal end removably connects to the proximal end of the catheter assembly and when pulled by the tunneler during tunneling, pushes the catheter assembly at its proximal end to follow the tunneler through the newly created tunnel.

The adapter includes a distal connector section and a locking section on the distal connector section that releasably locks to a connector section on the proximal tunneler end. The adapter's distal connector section and the tunneler's connector section are shaped and dimensioned to fit within the sheath when locked together and the sheath pulled thereover. The adapter has an elongated, small diameter cylindrical body that is insertable through the selected catheter lumen until its proximal end protrudes beyond the proximal end of the catheter assembly. A plug is releasably securable onto the protruding proximal end portion of the adapter's cylindrical body to establish a releasable connection to the proximal end of the catheter assembly. Preferably, a stylet is utilized with the catheter assembly and includes a proximal end with a tapered entrance into which the plug on the adapter is pressed for performance of the tunneling procedure, to secure against axial movement of the tunneler assembly with respect to the catheter assembly.

### BRIEF DESCRIPTION OF THE DRAWINGS

The accompanying drawings, which are incorporated herein and constitute part of this specification, illustrate the presently preferred embodiments of the invention, and, together with the general description given above and the detailed description given below, serve to explain the features of the invention. In the drawings:

FIG. 1 is a side profile view, partially in section, of a catheter tunneler assembly of the present invention, inserted into a catheter.

FIG. 2 is an enlarged portion of FIG. 1 showing a connection between a tunneler and adapter of the catheter tunneler assembly.

FIG. 3 is an enlarged side profile view of the catheter tunneler shown in FIG. 1.

FIG. 4 is an enlarged portion of FIG. 3, showing a proximal end of the tunneler.

FIG. 5 is an enlarged side profile view of the adapter of FIG. 2.

FIG. 6 is an end view of the adapter taken along lines 6-6 of FIG. 5.

FIG. 7 is an enlarged side profile view of a plug securable to the adapter of FIGS. 1 to 6; and

FIG. 8 is an end view of the plug taken along lines 8-8 of FIG. 7.

### DETAILED DESCRIPTION OF THE INVENTION

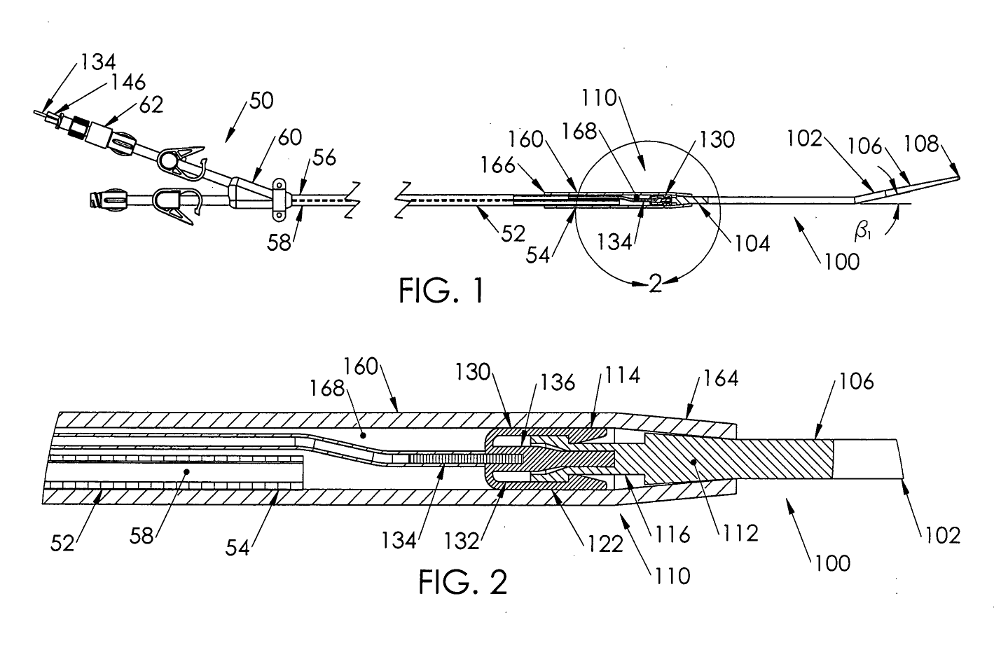

In the drawings, like numerals indicate like elements throughout. The terminology includes the words specifically mentioned, derivatives thereof and words of similar import. The embodiments illustrated below are not intended to be exhaustive or to limit the invention to the precise form disclosed. The word “proximal” as used herein, is defined to mean the direction closer to the end of a catheter that is connected to an external device, shown as the left hand side of the catheter 52 in FIG. 1. The word “distal” as used herein, is defined to mean the direction closer to the end of the catheter that is inserted into a patient's blood vessel, shown as the right hand side of the catheter 52 in FIG. 1. Regarding tunneler 102, the left hand side is denoted herein as the proximal end, and the right hand side as the distal end. These embodiments are chosen and described to best explain the principle of the invention and its application and practical use and to enable others skilled in the art to best utilize the invention.

Referring first to FIG. 1, catheter assembly 50 includes catheter 52 having at least one lumen and a distal catheter end 54, and a catheter tunneler assembly 100 according to the preferred embodiment of the present invention is shown secured to the distal catheter end 54. In FIG. 1, catheter 52 is shown having two lumens, an arterial lumen 56 and a venous lumen 58. The catheter assembly also is shown having a hub 60 that secures extension tubes to the catheter, the extension tubes respectively having clamps and luer caps thereon, and also a stylet 62 that is removably secured to the luer cap affixed to the proximal end of the arterial lumen 56.

Catheter tunneler assembly 100 is used to connect to the distal end of the catheter of catheter assembly 50 to pull it through a subcutaneous tunnel during catheter insertion into a patient. The tunneler assembly 100 includes a tunneler 102 and a connector assembly 110 on its proximal end 104.

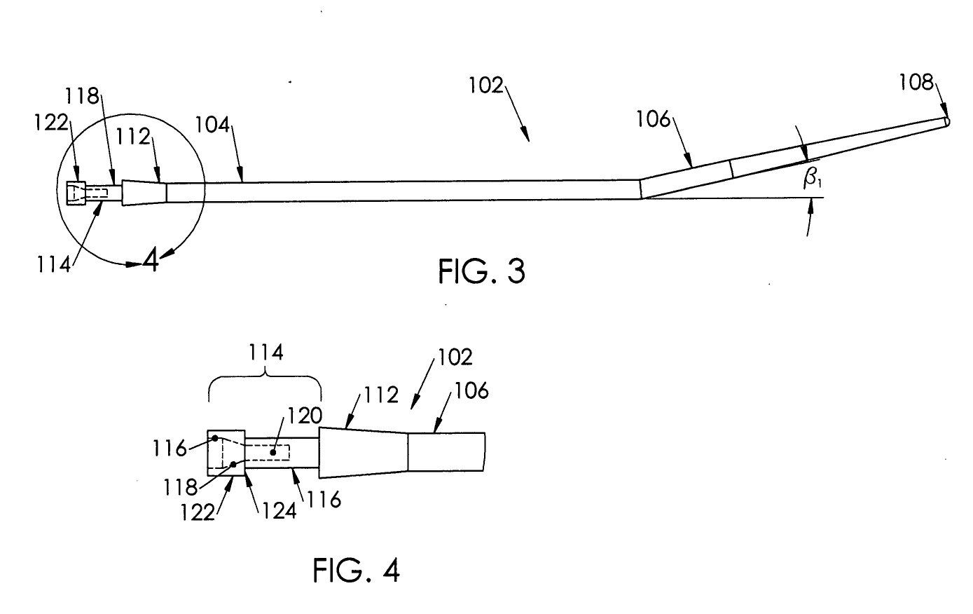

Referring now to FIGS. 1 and 3, the tunneler 102 includes an elongated, generally cylindrical body 106 having a distal tip 108. The body 106 preferably bends at a predetermined location along its length; as shown in FIGS. 1 and 3, the body 106 is bent at an angle β1 of approximately 12 degrees, although those skilled in the art will recognize that it may be bent more or less than 12 degrees or have no bend at all. The distal tip 108 is preferably blunt, and is inserted into the tunnel after a separate tunneling device (not shown) has been used to form the tunnel. Alternatively, those skilled in the art will recognize that the distal tip 108 may include a sharp point so that the tunneler 102 may be used to form the tunnel.

In reference to FIGS. 2 to 4, the proximal end 104 of the tunneler 102 includes a generally frusto-conical retainer 112 that is used to retain the sheath 160 onto the tunneler 102 during use. A connector section 114 extends proximally from the retainer 112 and is used to retain the adapter 130 onto the tunneler 102. The retainer 112 includes a cylindrical elongated member 114 having a passageway 116 extending partially thereinto that preferably includes a tapered portion 118 that extends at least partially through the passageway 116 and is shown to conclude in a narrowed inner portion 120. A proximal end of the elongated member 114 includes an enlarged portion 122 having a distal lip 124 that is used to connect the tunneler 102 to the adapter 130. Preferably, the tunneler 102 is constructed from stainless steel, although those skilled in the art will recognize that other suitable materials may be used.

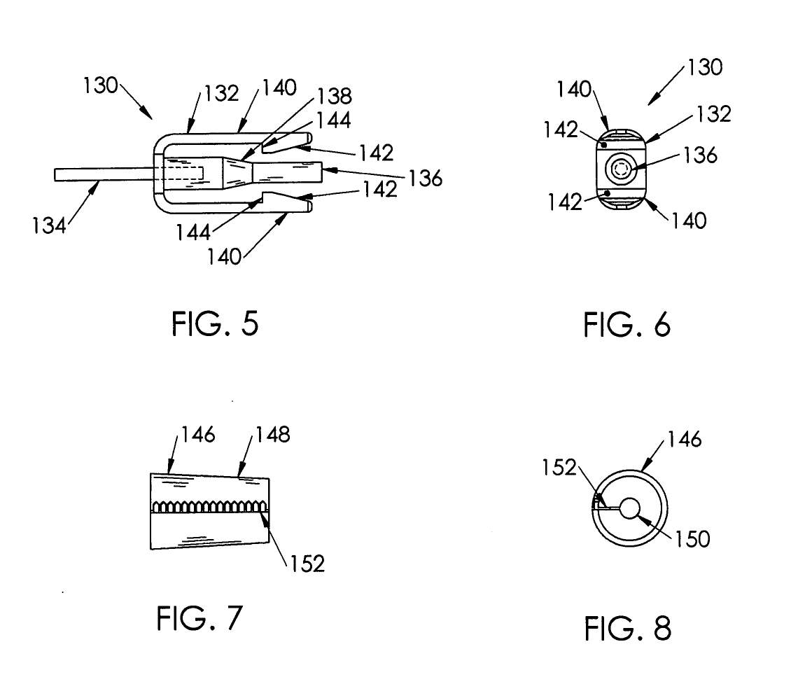

Referring to FIGS. 2, 5 and 6, the adapter 130 includes a distal portion 132, shown to be shaped in the general form of a trident. The distal portion 132 is fixedly connected to an elongated cylindrical body 134 and includes a central prong 136 that includes a tapered portion 138. The central prong 136 and the tapered portion 138 are sized to fit snugly within the passageway 122 in the tunneler 110.

The distal adapter portion 132 also includes a plurality of gripping fingers 140 that extend generally parallel to the central prong 136 and spaced laterally therefrom. Preferably, two gripping fingers 140 are used, although those skilled in the art will recognize that more than two gripping fingers 140 may be used. Each gripping finger 140 includes an inwardly facing tapered face 142 that extends proximally toward the central prong 132 for engaging and camming over enlarged portion 122 of tunneler connector section 114 which temporarily deflects the gripping fingers 140 outwardly to pass thereover. Each gripping finger 140 also includes a locking surface 144 that extends proximally of the tapered face 142 and is generally perpendicular to the central prong 136. Each locking surface 144 is adapted to lockingly engage with the distal lip 124 of the enlarged portion 122 (FIG. 4) on the tunneler 102 to retain the adapter 130 onto the tunneler.

While the gripping fingers 140 are preferably used to connect the adapter 130 to the tunneler 102, those skilled in the art will recognize that other connection methods, such as threaded connections, barbs, and other known connection means may be used to connect the adapter 130 to the tunneler 102.

The cylindrical body 134 is preferably connected to the distal portion 132 by solvent bonding, overmolding, or some other suitable method known by those skilled in the art. Preferably, the distal portion 132 is constructed from nylon or some other suitable material. The cylindrical body 134 is small in diameter and is preferably constructed from nylon or some other suitable material that allows the cylindrical body 134 to flex as the cylindrical body 134 is inserted through the selected lumen of catheter 52 from its distal tip until the proximal and exits from the proximal end of catheter assembly 50 beyond the luer cap on the extension sleeve for the selected lumen.

A plug 146 is shown in FIGS. 1, 7 and 8 and includes a generally frusto-conically shaped body 148 having a through passage 150 extending axially therethrough and having a slot 152 that extends the length of the body 148 from the through passage 150 to the exterior of the body 148. Slot 152 is sized to allow the cylindrical body 134 of the adapter 130 to be inserted thereinto from laterally thereof until seated within through passage 150.

Referring back to FIGS. 1 and 2, the sheath 160 includes a generally tubular sheath body 162 having a distal end 164 and a proximal end 166. A through passage 168 extends between the distal end 164 and the proximal end 166. The distal end 164 is tapered, with the through passage 168 having a corresponding taper. The taper in the through passage 168 corresponds with the frusto-conical retainer 112 so that the sheath 160 engages the retainer 112 and prevents the sheath 160 from further sliding proximally thereof. Additionally, the sheath body 162 is sufficiently long to completely encase the distal portion 132 of the adapter 130 as well as the distal ends of the catheter lumens in the catheter 52, as shown in FIG. 1. Preferably, the sheath 160 is constructed from polypropylene or some other suitable material.

In use, referring to FIG. 1, a stylet 62 is used with the catheter assembly and is inserted from the proximal end of the catheter assembly 50 into the arterial lumen 56. Preferably, the stylet 62 extends exterior of the distal end of the arterial lumen 56 and distally of the distal end of the venous lumen 58. The stylet 62 includes an axial passage extending therethrough that is sized to allow the cylindrical body 134 of the adapter 130 to pass therethrough. The cylindrical body 134 is sufficiently long such that the cylindrical body 134 extends through the stylet's axial passage and out the proximal end thereof.

The plug 146 is placed onto the proximal end of the cylindrical body 134 after the cylindrical body 134 is passed through the stylet's axial passage and the cylindrical body's proximal end portion protrudes beyond the stylet. The plug is then advanced and pressed into the stylet 62, compressing the slot 152 so that the plug 146 engages and restricts the cylindrical body 134 from movement. The slot 152 allows the plug 146 to be compressed to grip tightly the cylindrical body 134 of the adapter 130 when the plug 146 is inserted into the stylet 62. Preferably, the plug 146 is constructed from an elastomer, such as a KRATON® polymer, or some other suitable material that allows the plug 146 to deform under compression without fracture.

The connector section 114 (FIGS. 1 and 2) on the tunneler 102 is connected to the distal portion 132 of the adapter 130 by inserting the central prong 136 of the distal portion 132 into the passageway 116 in the connector section 114. The central prong 136 is advanced sufficiently into the connector section 114 so that the tapered portion 138 of the central prong 136 bottoms out against the tapered portion 118 of the passageway 116. Generally simultaneously, the tapered face 142 of each gripping finger 140 engages the enlarged portion 122 on the connector section 114, biasing the gripping fingers 140 outward. As the gripping face 144 of each gripping finger 140 passes distally of the distal lip 124, the gripping fingers 140 resile back toward their original undeflected positions, locking the tunneler 102 onto the adapter 130.

Next, the sheath 160 is first inserted over the distal tip 108 of the tunneler so that the distal tip 108 of the tunneler 102 enters the proximal end 162 of the sheath 160, passes through the through passage 168 and out the distal end 164 of the sheath 160. The sheath 160 is advanced proximally along the tunneler 102 until the retainer 112 engages the inside surface of the tapered distal end 164 of the sheath 160. Sheath 160 will hold the adapter latch arms in place locked to the tunneler proximal end during tunneling.

The catheter 52 is now ready to be tunneled according to known techniques with the plug of the adapter pushing the catheter assembly through the newly made tunneler and following the tunneler. After tunneling, the plug 146 is removed from the stylet 62, allowing the through passage 150 to open up and release the cylindrical body 134 of the adapter 130 whereafter the remaining part of the tunneler assembly 100 is advanced distally from the stylet 62 and the catheter 52 and discarded. Alternatively, the cylindrical adapter body 134 may be severed distally of the catheter's distal tip 54, and the plug 146 remains secured to the cylindrical body 134 proximal end portion and facilitates proximal removal of the cylindrical body from the catheter assembly. The catheter 52 may now be inserted into the patient's blood vessel according to known techniques.

While the catheter 52 shown in FIGS. 1 and 2 is a side-by-side dual lumen catheter, those skilled in the art will recognize that the tunneler assembly 100 of the present invention may be used with single lumen catheters and catheters having more than two lumens, as well as co-axial catheters.

While the above disclosure uses the stylet 62 to assist in the insertion of the catheter 52 into the patient, those skilled in the art will recognize that the stylet 62 may also be omitted, with the plug 146 being inserted directly into the cap on the proximal end of the extension tube for the arterial lumen 56.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claim.

What is claimed is:

1. A catheter tunneler assembly for use with a catheter assembly having a catheter with at least one lumen, comprising:
a tunneler having a proximal tunneler end and a distal tunneler end; and
a connector assembly removably securable to the proximal tunneler end and at least stoppingly engageable with the proximal end of the catheter assembly.

2. The catheter tunneler assembly of claim 1, wherein the connector assembly includes an adapter comprising a distal end and including the small diameter cylindrical body that concludes in a proximal connector that is at least stoppingly engageable with the catheter assembly proximal end, wherein the distal adapter end includes a distal connector, and the connector assembly further includes a corresponding connector section at the proximal tunneler end that is releasably connectable to distal connector of the adapter.

3. The catheter tunneler assembly of claim 2, further comprising a sheath having a distal end, a proximal end, and a passageway extending therethrough, wherein the sheath is adapted to slide over the tunneler and to encompass the proximal tunneler end portion, the adapter, and a portion of the distal end of the catheter of the catheter assembly.

4. The catheter tunneler assembly of claim 3, wherein the tunneler proximal end includes a retainer, and the sheath distal end stoppingly engages with the retainer in the sheath's desired position when the sheath is slid onto the tunneler.

5. The catheter tunneler assembly of claim 2, wherein the proximal connector is a plug having a tapered body and a plug passageway extending therethrough, wherein the plug passageway is sized for the to allow the elongated cylindrical body of the adapter to extend therethrough, and that is adapted to stoppingly engage with a proximal end of the catheter assembly.

6. The catheter tunneler assembly of claim 5, wherein the plug includes a longitudinal slot extending radially from the circumference of the plug to the passageway whereby a proximal end portion of the elongated cylindrical body is pressed laterally until seated within and along the passageway, whereafter the plug is insertable into a corresponding recess extending into a cap secured onto the catheter assembly at the proximal end thereof aligned with the or one lumen of the catheter, to stoppingly engage the catheter assembly whereafter the tunneling procedure may be performed.

7. The catheter assembly of claim 5, wherein a stylet is secured to the proximal end of the catheter assembly and includes a passage that extends into and along the or one lumen of the catheter through which the adapter cylindrical body extends, and the plug is insertable into and seats within a recess extending into a proximal end of the stylet and is compressed against the cylindrical body of the adapter that extends therethrough.

8. The catheter tunneler assembly of claim 5, wherein the tunneler's corresponding connector section includes a locking section and the distal adapter connector section includes a cooperating locking section whereby the adapter locks to the tunneler to secure the connector assembly to the tunneler in a manner permitting disconnection therefrom.

9. The catheter tunneler assembly of claim 8, wherein the locking section includes at least one latching ledge facing toward the tunneler distal end, and the cooperating locking section includes at least one cooperating latching ledge facing toward the catheter assembly.

10. The catheter tunneler assembly of claim 9, wherein the locking section includes a circumferential latching ledge facing toward the tunneler distal end, and the cooperating locking section includes at least one cooperating latching ledge facing toward the catheter assembly.

11. The catheter tunneler assembly of claim 10, wherein the at least one cooperating latching ledge is disposed on a free end of a respective latch arm of the adapter that extends toward the tunneler and is adapted to be deflected radially outwardly to pass over the latching ledge until resiling to its unstressed position when the cooperating latching ledge passes the latching ledge and latches therebehind, the latch arm being so shaped and dimensioned as to fit within the sheath passageway when latched into position with respect to the locking section.

12. The catheter tunneler assembly of claim 10, wherein the adapter's, distal connector includes at least two latch arms that extend toward the tunneler and conclude in free ends on which respective cooperating latching ledges are defined, the free ends being adapted to be deflected radially outwardly to pass over the latching ledge until resiling to their unstressed positions when the cooperating latching ledge passes the latching ledge and latches therebehind, the latch arms being so shaped and dimensioned as to fit within the sheath passageway when latched into position with respect to the locking section.
